(19) United States
(12) Patent Application Publication
(10) Pub. No.: US 2007/0104763 A1
(43) Pub. Date: May 10, 2007

(54) Composition of fentanyl citrate oral solid transmucosal dosage form, excipient and binding material therefore, and methods of making

(75) Inventors: Christopher N. Jobdevairakkam, Plainsboro, NJ (US); Benjamin Selvaraj, Plainsboro, NJ (US)

Correspondence Address: BRADLEY N. RUBEN, PC, 463 FIRST ST, SUITE 5A, HOBOKEN, NJ, 07030, US

(73) Assignee: Navinta LLC

(21) Appl. No.: 11/271,767

(22) Filed: Nov. 10, 2005

(51) Int. Cl. A61K 9/28; A61K 31/445; A61K 9/68

(52) U.S. Cl. 424/441; 514/317

(57) ABSTRACT

A process of preparing a highly uniform oral transmucosal lozenge of fentanyl citrate (a “fentanyl lollipop”) provides uniform distribution of the drug. The content uniformity between the lozenges and uniform distribution of the drug within a lozenge is achieved by dry mixing a micronized drug of a particle size of about one to ten microns with at least one major excipient, such as a dextrose, having cavities and pores on its surface after pressing into the lozenge shape. The major component of the lozenge can be a binding material prepared with a mixture of dextrose hydrate, food grade starch and water. This binding material has better strength to bind the stick to the lozenge due to stronger cross-linked matrix formation between the lozenge and the binding material.

### BACKGROUND OF THE INVENTION

1. The Field of the Invention

This invention relates to lozenges for drug delivery, including those with a holding implement (e.g., lollipops), a binder for the drug, and to method for making each of those.

2. The State of the Art

Opioids are the most oftenly administered analgesics that are both safe and effective. Opioids are found to be effective due to their ability to bind to specific receptors both within and outside the central nervous system. They are safe, and non-addicting, when pain is actually present. Fentanyl is one among the most commonly used opioids to manage moderate to severe pain such as neuropathic pain, cancer pain, and other chronic arthritic pain. Some opioid analgesics, such as fentanyl, have been administered through oral mucosal tissue. To produce a quality transmucosal fentanyl dosage form, several factors must be considered. There should be content uniformity among the single dosage units, uniform distribution of the drug within each single dosage unit, and the lozenge should have a uniformly integral structure. The dosage formulation must address these factors associated with the oral transmucosal delivery to produce effective transmucosal absorption.

Quite often the phrase “oral drug delivery” references drug absorption in the gastrointestinal tract via oral delivery. Oral delivery leading to absorption of the drug by oral mucosal tissues often referred as transmucosal delivery, has certain advantages. Oral transmucosal delivery permits the drug to be introduced across a mucous membrane, thereby avoiding the gastrointestinal tract and introducing the drug directly into the circulation. Another advantage of oral transmucosal delivery is that it is a non-invasive drug delivery method with a high level of patient compliance.

Lozenges, chewing gums, and tablets have all been used for oral transmucosal delivery of pharmaceuticals. Sublingual tablets are designed to deliver small amounts of a potent drug, which is almost immediately dissolved and absorbed. U.S. Pat. No. 5,711,961 to Reiner, et al. discloses a chewing gum for the delivery of pharmaceuticals. The chewing gum delivery dosage form of Reiner is primarily directed for patients who may be more disposed to self-administer a drug in chewing gum form as opposed to other less familiar dosage forms. U.S. Pat. No. 5,298,256 to Flockhart et al. discloses an oral transmucosal delivery using a buccal patch.

Most lozenges or tablets are typically designed to dissolve in the mouth over several minutes. This allows extended dissolution of the lozenge and absorption of the drug. A lozenge-on-a-handle dosage form of transmucosal drug delivery of Fentanyl is disclosed in U.S. Pat. Nos. 4,671,953, 5,132,114, 5,288,497, 5,855,908, and 5,785,989, all to Stanley et al. These patents describe methods for producing solid dosage forms containing a drug in a dissolvable sugar-based matrix. One method achieves a solid dosage form by mixing the drug into a molten sugar base and allowing the base to solidify into a hard candy. Another method describes compressing a powder, in which the drug has been well-dispersed, into a solid dosage form.

The bitterness or other unpleasant taste of the drug is masked by the large amounts of sugar added to lozenge and lollipop delivery devices. Flavor enhancers or other sweeteners may also be included to provide an organoleptically satisfactory product. An FDA-approved lozenge-on-a-handle-type oral transmucosal solid dosage form containing fentanyl is marketed in the US under the mark ACTIQ by Cephalon Inc. ACTIQ is available in several strengths ranging from 200 μg to 1600 μg single dosage units. ACTIQ contains a matrix composed of hydrated dextrose, confectionary sugar, and starch. In the ACTIQ lollipop or lozenge-on-a-handle, the handle is fixed to the matrix using a food grade starch base as the binding material.

Fentanyl is a very powerful narcotic analgesic and hence requires a very consistent and uniform dosage form formulation procedure. Therefore, it is worth developing a formulation process for making microgram dosage units enabling the production of final oral dosage units that contain a consistent quantity of the active substance dispersed uniformly in the dosage unit, and to provide similar uniformity among multiple dosage units manufactured.

The aforementioned '953 patent discloses the concept of making an oral dosage form suitable for diabetic patients employing sorbitol or mannitol and using artificial sweeteners such as aspartames. A similar sugar free transmucosal solid dosage form using a polyhydric alcohols matrix has been described in US patent application 20040253307 to Hague.

The prior art has disclosed various compositions of a solid oral transmucosal dosage form. US Patent Application 20040092531, Chiszh, et al., discloses a combination of active ingredients containing at least one opioid compound with a fentanyl-type structure and ketamine. The application discloses a weight ratio of active substance component a to active substance component b in the range of 1:20 to 1:1500. US PatentApplication 20020160991, Shao, discloses compositions of orally bioavailable formulations of fentanyl and its congeners and an excipient selected from the group consisting of cyclodextrins, liposomes, micelle forming agents, and polymeric carriers.

The aforementioned patents to Stanley et al. and the application to Hague disclose the composition of an oral transmucosal dosage form and the composition of a fentanyl containing lozenge (lollipop). However, the process of preparing the fentanyl lollipop is not been disclosed. The binding material composition is an important factor because it determines the strength of the oral transmucosal lozenge (lollipop).

The Stanley et al. '989 patent reveals that a dissolvable compressed matrix may be attached to a holder, such as a handle. The holder may be glued to the matrix by dissolvable adhesive such as confectioner's glue, liquid sorbitol, or wax. Alternatively, the holder may be compressed, screwed, snapped, or molded into the dissolvable matrix as described above, or a dissolvable matrix may be sprayed or otherwise deposited onto a handle during formation. The dissolvable matrix may also be formed around an insert onto which a holder can be attached.

The Hague '307 application discloses the use of a food-grade glue to assemble a holder with a compressed sugar-free fentanyl citrate, where the matrix is described as primarily Purity Gum BE (also known as E1450 starch, starch sodium octenyl succinate), Confectioner's sugar, and purified water components. This is problematic since glue made out of sucrose or confectionary sugar requires a relatively long time and has low binding strength.

US Patent Application 20050079138, Chickering, discloses a method for making a dry powder blend pharmaceutical formulation where jet milling is utilized to create improved dispersibility, suspendability, or wettability of the microparticles when blended with an excipient. The milling process of the blend provides a uniform solid dosage form.

U.S. Pat. No. 6,908,626, Cooper et al., discloses a methodology for preparing uniform solid dosage forms comprising (1) particles of at least one poorly soluble nanoparticulate active agent, (2) at least one surface stabilizer adsorbed onto the surface of the nanoparticulate active agent particles, and (3) at least one poorly soluble microparticulate active agent, which can be the same as or different from the active agent (1). This process involves preparing solid dosage forms of a poorly soluble matrix by mixing nanoparticles and microparticles.

Bredenberg, et al., (Eur. J. Pharma. Sci., 20, 2003, 327-334) have reported dry blending a formulation for rapidly absorbed small sublingual fentanyl tablets. Fentanyl content in the tablet, with a mean weight of approximately 70 mg, is 0.9% for the 400 μg dosage, with a content uniformity of about 88 to 94%. It was reported that the average content of fentanyl is about 96% for the tablet weight of about 70 mg prepared by direct compression of dry blend of mannitol with fentanyl citrate of a calculated particle size of about 1 μm (surface area 2.3 m²/g). They have concluded that minor segregation had occurred during tablet processing.

In a fentanyl lollipop, the weight ratio of single dosage unit to drug is about 10,000 for a 200 μg dosage unit and about 1250 for a 1600 μg dosage unit. Content uniformity and the distribution of the drug within the matrix in such dosage forms having a high ratio of excipient(s) to drug is a difficult task. In such formulations, although the uniformity among lozenges might be achieved, non-uniformity within the lozenge often results, with concomitant variation in the peak drug absorption values( Cₘₐₓ). This variation within the dosage form (hot spots) could result in poor efficacy and safety. For a typical solid dosage form containing highly potent drugs such as fentanyl, the amount of drug released during any given time interval is very important, and the release rate should be uniform among all dosage units.

The foregoing publications are incorporated herein by reference.

### SUMMARY OF THE INVENTION

The present invention discloses achieving a uniform drug distribution within the matrix of a solid lozenge dosage form using a dry blending process and a particular excipient. The novel process allows preparation of such lozenges containing an active drug at a level of about 100 μg to about 3000 μg per single dosage unit, with better content uniformity of the drug among individual dosage units as well as within the matrix of a single dosage lozenge. The content uniformity of the drug among the lozenges is measured by assay of the drug in the lozenge by high performance liquid chromatography (hereinafter “HPLC”) whereas the uniform distribution of the drug within a lozenge is evidenced by the content of drug released in each coaxial quadrant of the lozenge. The lozenge prepared by the process of this invention is found to release the drug in a uniform dissolution rate measured by the ratio of drug to the major component (excipient) of the lozenge. The ratio of the release of the drug to the release of the major excipient is maintained constant. A new composition of a binding material, useful to fix the stick or holder to the drug-containing lozenge, is also provided. Further, a novel composition of pharmaceutical food grade binding material containing hydrated dextrose is revealed.

This invention discloses an oral transmucosal lozenge having uniform a distribution of fentanyl within a single dosage. Micronised fentanyl citrate of average particle size approximately 1 to 5 μm in diameter are found to be self-aggregated. Blending the micronized fentanyl citrate particles with a pharmaceutical excipient, such as hydrated dextrose, yields a blend wherein the fentanyl citrate particles are uniformly distributed over the surface of the dextrose particle. The energy of adsorption of fentanyl over the surface of the excipient is sufficient to break self-aggregation.

This invention also provides a new composition for a binding material or glue that contains at least one of the major components of the lozenge matrix. The presence of at least one ingredient in common between the lozenge matrix and the glue establishes better strength to the glue for binding the stick to the lozenge by cross linked crystallization. Moreover, a glue containing hydrated dextrose has the property of setting or curing without contraction in its original volume.

### BRIEF DESCRIPTION OF THE DRAWINGS

FIG. 1 shows the final lollipop and the quadrant areas used in the testing.

### DETAILED DESCRIPTION OF SPECIFIC EMBODIMENTS

Oral transmucosal administration of fentanyl citrate requires a precise and consistent formulation technique to address pharmacologically acceptable uniform drug content among multiple single dosage units and within a single dosage unit. The lozenge is made of a soluble sugar matrix, at least one buffer, at least one pharmaceutical agent, and the drug. The lozenge provides better absorption of the drug through the oral mucosa. Better absorption of a drug uniformly dispersed in a dosage unit is desirable for maintaining a reproducible dose. In addition to content uniformity, a strong binding of the stick to the lozenge (i.e., for a lollipop) must be maintained throughout the entire period of dosing.

One embodiment of the current invention provides a technique to achieve better content uniformity between the lozenges and a uniform drug distribution within the single dosage form. Micronized fentanyl citrate with an average particle diameter of about 1 μm to about 5μm was found to be self-aggregating. Particles of micronized fentanyl citrate, when blended with a pharmaceutically acceptable excipient such as hydrated dextrose, yield a matrix wherein the fentanyl citrate particles are uniformly distributed over the surface of the dextrose particles. The adsorption energy of fentanyl over the surface of the excipient likely enables the particle to break self-aggregation and become pharmacologically available.

According to another embodiment of this invention provided herein, a new composition of a binding material or glue contains at least one of the major components of the lozenge matrix. The presence of at least one ingredient in common between the lozenge matrix and the glue yields better strength in binding the stick to the lozenge by cross-linked crystallization. Moreover, the glue containing hydrated dextrose has the property of setting or curing without significant contraction from its original liquid volume.

The lozenge is made by compressing a dry blend containing the drug and excipient. The drug has been found by scanning electron microscopy to be adsorbed into cavities or pores present on the surface of the excipient. The average pore diameter of porous hydrated dextrose is approximately 3 μm to 20 μm and therefore is sufficient to hold the 1 μm to 10 μm drug particles obtained by micronization. One of the prime requirements for better uniformity requires the average particle diameter of the drug to be equal to or less than the surface cavity or pore diameter of the substrate. Fentanyl citrate is a crystalline material. Upon micronization, the fentanyl citrate becomes fine particles, approximately 1 μm to 10 μm in diameter. Preferably, after micronization, 100% by weight of the particles are less than about 20 μm, more preferably less then about 17 μm., 90% by weight of the particles are less than about 10 μm, more preferably less than about 7.5 μm, and 50% by weight is less than about 5 μm, more preferably less than about 3 μm. Strong interparticle forces induced by the milling lead to aggregation of the fentanyl citrate particles. Intimate blending of hydrated dextrose and micronized fentanyl citrate acts to physically disaggregate the fentanyl citrate particles from each other and allow them to be dispersed throughout the mixture.

The content uniformity of the drug among different lozenges was measured by assay of the drug in the lozenge by HPLC. A relatively uniform distribution of the drug within a lozenge is evidenced by the content of drug released in each coaxial quadrant of the lozenge. A lozenge prepared by the process of this invention was found to release the drug at a uniform dissolution rate (measured by the ratio of drug to the major component of lozenge). The content of fentanyl was determined by HPLC, using an ultraviolet (hereinafter “UV”) detector. The dextrose was determined by HPLC with a refractive index detector.

A process of preparation of the glue is also disclosed. This process comprises, dissolving a suitable quantity of hydrated dextrose in water at a temperature between about 50° C. and about 100° C., adding corn starch, and then cooling to a desired temperature between about 40° C. and about 70° C. The glue may be diluted with water either before or after cooling the mass. The glue prepared by this process has an insignificant volume change upon cooling, thereby reducing stresses between the glue, lozenge, and stick, maintaining the same surface area of contact, and thus providing a better bond.

Further details of this invention are demonstrated by the examples furnished herein. In these examples, all times, temperatures, and amounts are exact to a certain degree, and also have an error of a certain degree, both as would generally be expected for these types of experiments. In all of the experiments, the fentanyl citrate was micronized by milling, such as a jet mill, although other methods of milling, and other methods of particle preparation, are suitable and within the scope of this invention. Blending of dry ingredients is preferably accomplished in a V-blender, as in the examples, although other blending methods are contemplated by this invention. The combination of disodium hydrogen phosphate and citric acid in the examples is a conventional buffer combination. Although fentanyl citrate is used as the active ingredient in the examples, it should be appreciated that other active ingredient can be provided as lozenges using the present invention. It has been found that the glue can be delivered by gravity, or forced through the orifice of a dispensing nozzle or other dispensing device. In the examples below, 100% of the micronized particles were less than 17.3 μm, 90% by weight were less than 7.5 μm, and 50% by weight were less than 3.4 μm.

As is conventional in tabletting a dry composition, an excipient (non-active ingredient) is used as the carrier or matrix material. Other adjuvants, such as disintegrants, glidants, diluents, and/or lubricants, may also be present, as well as the more conventional colorants, flavorings, sweeteners, and other organoleptically-effecting materials.

The particular excipient and binder is dextrose monohydrate (hydrated dextrose). Powdered dextrose is crystallized dextrose hydrate, 9% water; anhydrous dextrose has less than 0.5% water. Dextrose hydrate is about 75% as sweet as anhydrous. U.S. Pat. No. 6,682,432 discloses a process for making dextrose monohydrate. Examples of binders and excipients usually include sugars, sugar alcohols, and mixtures thereof, including dextrose hydrate (such as Cerelose 2043).

Although this invention has been exemplified by fentanyl citrate, in general, this invention provides a technique to achieve better drug content uniformity between the dosage forms and a uniform drug distribution within the single dosage form. Activie ingredients such as drugs to which this invention is applicable include, without limitation, fentanyl, sufentanyl, remifentanyl, antidepressants (e.g., nefopam, oxypertine, doxepin, amoxapine, trazodone, amitriptyline, maprotiline, phenelzine, desipramine, nortriptyline, tranylcypromine, fluoxetine, imipramine, imipramine pamoate, isocarboxazid, trimipramine, and protriptyline,); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, prednisone, methylprednisolone acetate suspension, triamcinolone acetonide, methylprednisolone, prednisolone sodium phosphate, methylprednisolone sodium succinate, hydrocortisone sodium succinate, triamcinolone.hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium).

### EXAMPLE 1

### Preparation of Blend for 200 μg Dosage Unit

Approximately 252 mg of micronized fentanyl citrate having an average particle diameter of 3 μm was added to 1312 g of EMDEX (hydrated dextrose (dextrose monohydrate) containing about 7% maltodextrin, registered trademark of Edward Mendell Co., Inc., Patterson, N.Y., for spray-crystallized maltose-dextrose porous spheres for the production of compressed tablets, available from JRS Pharma LP, Patterson, N.Y.) and blended in a V-blender for about 5 min. Approximately 12.88 g of disodium hydrogen phosphate, 6.16 g of citric acid, 3.2 g flavor, and 249.5 g confectionary sugar 6× were then added. The combination was blended for 15 minutes. Approximately 16.0 g of magnesium stearate was then added and the mixture blended for 3 minutes to create a powdered product.

### EXAMPLE 2

### Preparation of Blend for 400 μg Dosage Unit

Approximately 503 mg micronized fentanyl citrate having an average particle diameter of about 3 μm was added to 1312 g of EMDEX brand maltose-dextrose and blended in a V-blender for about 5 min. Approximately 12.88 g of disodium hydrogen phosphate, 6.16 g of citric acid, 3.2 g flavor, 249.27 g confectionary sugar 6× were then added. The combination was blended for about 15 min. Approximately 16.0 g of magnesium stearate was then added and blended for about 3 minutes to create a powdered product.

### EXAMPLE 3

### Preparation of Blend for 1600 μg Dosage Unit

Approximately 1006 mg of micronized fentanyl citrate having an average particle diameter of about 3 μm was added to 1312 g of EMDEX brand maltose-dextrose and blended in a V-blender for about 5 min. Approximately 12.88 g of disodium hydrogen phosphate, 6.16 g of citric acid, 3.2 g flavor, 123.3 g confectionary sugar 6× were then added. The combination was blended for about 15 min. Approximately 16.0 g of magnesium stearate was added and blended for about 3 minutes to create a powdered product.

### EXAMPLE 4

### Preparation of Blend for 1600 μg Dosage Unit

Approximately 1006 mg of micronized fentanyl citrate of average particle diameter of about 3 μm is added to 1312 g of EMDEX brand maltose-dextrose and blended in a V-blender for about 5 min. Approximately 12.88 g of disodium hydrogen phosphate, 6.16 g of citric acid, 3.2 g flavor, 123.3 g confectionary sugar 6× were then added. The combination was blended for about 15 min. Approximately 16.0 g of magnesium stearate was added and blended for about 3 minutes to create a powdered product.

### EXAMPLE 5

### Preparation of Lozenge

Approximately 2.00 g of each blend was separately transferred to the die of a conventional tabletting machine over the lower punch and compressed with an upper punch to approximately 0.7 metric tons. The die cavity contained a mandrel that created a groove in the expelled product having dimensions suitable for a holder to be affixed in the manufacture of a lollipop. The resulting tablet was an oblong-shaped lozenge with a flattened side that contained a cavity. The side was flattened and given a cavity to facilitate attaching a holder with binding material.

### EXAMPLE 6

### Preparation of Binding Material (Glue)

Approximately 95 g of EMDEX brand maltose-dextrose was suspended in 35 mL of water and heated to approximately 65° C. for about 10 min. The temperature was increased, allowing the solution to boil, until a clear solution was obtained. Approximately 5 g of cornstarch 78-1551 (CAS 9005-25-8, a pregelatinized food grade starch available from National Starch and Chemical Co., Bridgewater, N.J.) was slowly added to the hot solution with stirring over a period of about 3 minutes. The mass was brought to a boil that was maintained for approximately one minute. The pasty mass was then cooled and preserved at about 65° C. for further usage.

### EXAMPLE 7

### Preparation of Binding Material (Glue)

Approximately 95 g of dextrose monohydrate was suspended in 35 mL of water and heated to approximately 65° C. for about 10 min. The temperature was then increased, allowing the solution to boil, until a clear solution was obtained. Approximately 5 g of cornstarch 78-1551 was slowly added and stirred for a period of about 3 minutes. The mass was then heated to a boil, which was maintained for approximately one minute. The pasty mass was then cooled and preserved at about 65° C. for further usage. The material prepared by this process was found to be as effective as the material prepared utilizing the method described in Example 6. Accordingly, at least about 75 wt. % dextrose monohydrate, more preferably about 88 wt. % dextrose monohydrate, and most preferably about 95 wt. % of the carbohydrate present in the binding material is dextrose monohydrate.

### EXAMPLE 8

### Preparation of Lollipop

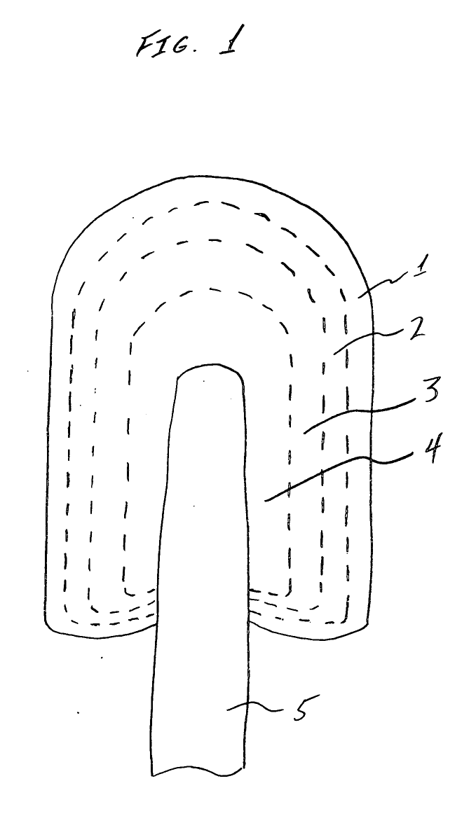

Various lozenges were arranged on a pallet, and approximately 50 μL of the glue prepared by the processes described in each Examples 6 and 7 was dispensed into the cavity of each lozenge. A stick was then inserted into the cavity containing the glue. The combination of glue, lozenge and stick was packed and allowed to cure at room temperature for about 10 hours; thus, the curing was by evaporation of the water. The result was a lollipop as shown in FIG. 1.

### EXAMPLE 9

### Testing of Fentanyl Uniformity within the Lozenge and Between Lozenges

The lollipops made by Example 8 were subjected to a dissolution test. The content of dextrose and drug released at different intervals of time was determined by a separate HPLC method. The lollipop was viewed as having four separate quadrants. As shown in FIG. 1, a plan view of a theoretical lollipop, the first quadrant represents the outermost approximately 25% by weight of the prolate hemispherical lollipop including the flat surface. The fourth quadrant represents the innermost approximately 25% by weight of the prolate hemispherical lollipop also including the flat surface. The results listed below are based on weight, not volume, so FIG. 1 is not to scale. The percentage of fentanyl released in each quadrant of the lollipop at levels of about 25%, 50%, 75% and 100% release of dextrose were determined for six samples, the results of which are provided in Tables 1 and 2. The ratio of the weight of fentanyl to the weight of dextrose at a particular interval of time was taken as a measure of the uniformity of the dissolution pattern of the lozenge; these results are shown in Tables 3 and 4. The content uniformity between the lozenges was determined by testing ten samples at each dosage level made in Examples 1-4; these results are furnished in Table 5.

TABLE 1
Fentanyl uniformity within the lollipop (dosage strength 400 μg)

Percentage of fentanyl in
each quadrant in six trials

| Quadrant | 1 | 2 | 3 | 4 | 5 | 6 |
|---|---|---|---|---|---|---|
| 1^{st} | 23 | 24 | 25 | 20 | 24 | 20 |
| 2^{nd} | 27 | 21 | 20 | 23 | 24 | 23 |
| 3^{rd} | 24 | 21 | 25 | 24 | 25 | 22 |
| 4^{th} | 20 | 21 | 20 | 26 | 24 | 28 |

TABLE 2
Fentanyl uniformity within the lollipop (dosage strength 1600 μg)

Percentage of fentanyl in
each quadrant in six trials

| Quadrant | 1 | 2 | 3 | 4 | 5 | 6 |
|---|---|---|---|---|---|---|
| 1^{st} | 24 | 23 | 27 | 28 | 27 | 24 |
| 2^{nd} | 23 | 27 | 23 | 20 | 24 | 28 |
| 3^{rd} | 27 | 26 | 21 | 25 | 25 | 24 |
| 4^{th} | 22 | 21 | 24 | 21 | 20 | 21 |

TABLE 3
Fentanyl and dextrose release data (dosage strength 400 μg)

Ratio of Fentanyl to Dextrose for six trials

| Time, min | 1 | 2 | 3 | 4 | 5 | 6 |
|---|---|---|---|---|---|---|
| 2 | 0.90 | 0.93 | 0.97 | 0.79 | 0.97 | 0.80 |
| 4 | 1.04 | 0.84 | 0.86 | 0.91 | 1.03 | 0.89 |
| 10 | 0.95 | 0.91 | 0.91 | 0.90 | 0.94 | 0.75 |
| 15 | 0.92 | 0.87 | 0.94 | 0.94 | 1.00 | 0.95 |
| 20 | 0.91 | 0.88 | 0.86 | 0.99 | 0.97 | 0.92 |
| 30 | 0.95 | 0.91 | 0.87 | 0.95 | 0.97 | 0.91 |
| 45 | 0.91 | 0.92 | 0.98 | 0.90 | 0.97 | 0.92 |

TABLE 4
Fentanyl and dextrose release data (dosage strength 1600 μg)

Ratio of Fentanyl to Dextrose for six trials

| Time, min | 1 | 2 | 3 | 4 | 5 | 6 |
|---|---|---|---|---|---|---|
| 2 | 0.96 | 0.95 | 1.06 | 0.95 | 0.94 | 0.97 |
| 4 | 0.95 | 0.98 | 0.96 | 1.01 | 0.99 | 1.00 |
| 10 | 0.97 | 0.97 | 0.94 | 0.96 | 0.99 | 0.99 |
| 15 | 0.99 | 0.98 | 0.97 | 1.00 | 0.98 | 0.99 |
| 20 | 0.99 | 0.97 | 0.96 | 0.98 | 0.97 | 0.98 |
| 30 | 0.99 | 0.94 | 0.96 | 0.97 | 0.98 | 0.97 |
| 45 | 0.97 | 0.94 | 0.93 | 0.95 | 0.96 | 0.97 |

TABLE 5
Content uniformity of fentanyl lozenges

| Dosage | Range, % | Average | % RSD |
|---|---|---|---|
| 200 μg | 98.8-101.6 | 100.8 | 0.9 |
| 400 μg | 98.4-102.5 | 100.7 | 1.6 |
| 1600 μg | 95.3-102.0 | 98.4 | 2.7 |

### EXAMPLE 10

### Preparation of Blend for 400 μg Dosage Unit

Approximately 503 mg of micronized fentanyl citrate of average particle diameter of 3 μm was added to 1312 g of dextrose monohydrate and blended in a V-blender for 5 min Approximately 12.88 g of disodium hydrogen phosphate, 6.16 g of citric acid, 3.2 g flavor, 249.27 g confectionary sugar 6× was added. The combination was blended for about 15 min. Approximately 16.0 g of magnesium stearate was added and blended for 3 minutes.

### EXAMPLE 11

### Preparation of Blend for 1600 μg Dosage Unit

Approximately 1006 mg of micronized fentanyl citrate of average particle diameter of 3 μm was added to 1312 g of EMDEX brand maltose-dextrose in aliquots and blended in a V-blender for about 5 min each. Approximately 12.88 g of disodium hydrogen phosphate, 6.16 g of citric acid, 3.2 g flavor, 123.3 g confectionary sugar 6× were added. The combination was blended for about 15 min. Approximately 16.0 g of magnesium stearate was added and blended for about 3 minutes.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A lozenge composition, comprising: a predetermined amount of a micronized Fentanyl salt dispersed uniformly throughout an excipient matrix comprising as its major component dextrose monohydrate.

2. The lozenge of claim 1, wherein the lozenge is in the form of a lollipop having a holder glued to the lozenge.

3. The lozenge of claim 2, wherein the glue is predominantly dextrose.

4. The lozenge of claim 1, wherein the dose of Fentanyl ranges from about 200 μg to about 1600 μg in the lozenge.

5. The lozenge of claim 2, wherein the dose of Fentanyl ranges from about 200 μg to about 1600 μg in the lozenge.

6. The lozenge of claim 1, further comprising a buffer.

7. The lozenge of claim 2, further comprising a buffer.

8. The lozenge of claim 6, wherein the buffer is a combination of disodium hydrogen phosphate and citric acid.

9. The lozenge of claim 7, wherein the buffer is a combination of disodium hydrogen phosphate and citric acid.

10. A lozenge produced by the process comprising: micronizing a Fentanyl salt; dry blending a predetermined amount of the micronized Fentanyl salt with a predetermined amount of an excipient having dextrose monohydrate as its major component to produce a mixed blend; and compressing the mixed blend into a lozenge.

11. The lozenge of claim 10, wherein the dry blending further comprises the addition of at least one ingredient selected from the group consisting of buffers, additional binders, lubricants, disintegrants, glidants, diluents, lubricants, colorants, flavorings, and sweeteners, and compatible mixtures thereof.

12. The lozenge of claim 10, wherein the amount of the Fentanyl salt is chosen to provide a lozenge dosage of between about 200 μg to about 1600 μg.

13. The lozenge of claim 10, wherein the process further comprises providing a holder; and gluing the holder to the lozenge.

14. The lozenge of claim 13, wherein the glue comprises primarily dextrose.

15. A process for making a pharmaceutically acceptable glue for a solid oral dosage form, comprising: suspending a predetermined amount of dextrose monohydrate to water in hot water; elevating the water temperature to boiling and maintaining an elevated temperature until a hot clear solution is obtained; mixing into the hot clear solution a food grade starch; and cooling the resultant glue mass.

16. The process of claim 15, wherein the dextrose monohydrate comprises at least about 75 wt. % of the dry ingredients.

17. The process of claim 16, wherein the dextrose monohydrate comprises at least about 88 wt. % of the dry ingredients.

18. A pharmaceutically acceptable glue for a solid oral dosage form, produced by the process comprising: suspending a predetermined amount of dextrose monohydrate to water in hot water; elevating the water temperature to boiling and maintaining an elevated temperature until a hot clear solution is obtained; mixing into the hot clear solution a food grade starch; and cooling the resultant glue mass.

19. A sold oral dosage form lozenge, wherein the improvement comprises the combination of micronized Fentanyl citrate particles in combination with an excipient being predominantly dextrose monohydrate.

20. The lozenge of claim 19, wherein the excipient further comprises up to about 10 wt. % maltodextrin.

21. The lozenge of claim 19, wherein the lozenge further comprises a holder, and wherein a further improved comprises adhering the holder to the lozenge with a binder consisting essentially of dextrose monohydrate with a minor portion of a starch.

22. An oral transmucosal lozenge defined by first to fourth volumetric quadrants each quadrant spatially outside the previous quadrant, and each quadrant having approximately the same weight as each other quadrant, said lozenge having dispersed throughout an active ingredient, wherein the active ingredient is present in each quadrant in an amount of about 20% to 30% of the total amount of active ingredient present.

23. The lozenge of claim 22, wherein the active ingredient is selected from the group consisting of analgesics, antidepressants, antihypertensive agents, antianxiety agents, steroidal compounds and hormones, and compatible combinations thereof.

24. The lozenge of claim 23, wherein the analgesic is selected from the group consisting of fentanyl, sufentanyl, remifentanyl, and compatible mixtures thereof.

25. The lozenge of claim 24, further comprising a holder attached to the lozenge to provide a lozenge in the form of a lollipop.

26. The lozenge of claim 25, wherein the holder is attached with dextrose.
